(19) United States
(12) Patent Application Publication
(10) Pub. No.: US 2006/0067900 A1
(43) Pub. Date: Mar. 30, 2006

(54) Method and composition for imparting high shine to a polymeric substrate

(75) Inventors: Steven H. Bertz, Morristown, NJ (US); Steven A. Orofino, Hamburg, NJ (US); Ellen S. Botschka, Riverdale, NJ (US)

Correspondence Address: William J. Davis, Esq.;INTERNATIONAL SPECIALTY PRODUCTION, Legal Department, 1361 Alps Road, Building No. 8, Wayne, NJ, 07470, US

(73) Assignee: ISP INVESTMENTS INC.

(21) Appl. No.: 10/952,948

(22) Filed: Sep. 29, 2004

(51) Int. Cl. A61K 8/39; C09G 1/02

(52) U.S. Cl. 424/64; 106/3

(57) ABSTRACT

A method and composition for imparting high shine to a polymeric substrate such as hair or skin, leather or a plastic which comprises applying thereto a phenylethyl or benzyl ester.

### CROSS-REFERENCE TO RELATED U.S. PATENT APPLICATIONS

This application is related to co-pending U.S. patent applications Ser. No. 10/859,533; filed Jun. 2, 2004; which is a continuation-in-part of co-pending U.S. application Ser. No. 10/617,407; filed Jul. 11, 2003, which are assigned to the same assignee as herein, the entire contents of which are hereby incorporated by reference.

### BACKGROUND OF THE INVENTION

1. Field of the Invention

This invention relates to a method and composition for imparting high shine to a polymeric substrate such as hair or skin, leather or plastic.

2. Description of the Prior Art

Personal care products are used to give polymeric substrates such as hair and skin a desired property such as body, hold or color. However, it is also desirable for such products to impart shine or gloss (luster) to hair or skin. For example, a fixative hair composition should be capable of adding luster to treated hair; and a color cosmetic such as lipstick should impart shine to the lips of the user.

Leather, imitation leather and other plastics often find high shine a desirable appearance.

Accordingly, it is an object of this invention to provide a method and composition for imparting high shine, gloss or luster to a polymeric substrate, such as hair or skin, leather, imitation leather or other plastics.

### SUMMARY OF THE INVENTION

What is described herein is a method of imparting high shine to a polymeric substrate such as hair or skin, leather or plastic, which comprises applying thereto a phenylethyl ester. The invention particularly includes compositions for imparting the desired high shine, gloss or luster to hair or skin.

Suitable esters used here include phenylethyl, benzyl or substituted benzyl esters, which are aryl carboxylic esters of 2-phenylethyl alcohol, 1-phenylethyl alcohol, benzyl alcohol or substituted benzyl alcohol. Representative esters include 2-phenylethyl benzoate, 2-phenylethyl toluate, di-2-phenylethyl phthalate, 1-phenylethyl benzoate, benzyl benzoate and substituted benzyl benzoates. A preferred phenylethyl ester is 2-phenylethyl benzoate (X-TEND™ 226, ISP). Most preferably, the refractive index of the ester is about 1.5.

In this invention, the spectral reflections of light on hair or skin, or natural or synthetic leather or plastics are substantially enhanced by the presence of the phenylethyl ester thereon.

Typical personal care formulations are color cosmetics, a lipstick, or a hair care product.

### DETAILED DESCRIPTION OF THE INVENTION

The advantageous property of X-TEND™ 226 to solubilize organic compounds, particularly solid organic sunscreen agents such as avobenzone and oxybenzone, and to provide low residue or non-whitening antiperspirant compositions is described in the related patent applications listed above.

What has been discovered herein is that such esters also can impart shine, gloss and luster to polymeric substrates such as hair or skin, or leather or imitation leather and other plastics, by application of X-TEND™ 226 to the substrate, preferably in a hair fixative composition or a lipstick. It is believed that these properties are achieved because of the relatively high refractive index of X-TEND™ 226, which is about 1.5.

Suitably, the ester is present in a composition in an amount sufficient to impart the desired shine to hair or skin, generally in an amount of about 0.1-25% by weight of the composition, preferably 1 -10%.

The invention will now be described in detail with reference to the following examples, in which:

### EXAMPLE 1

The formulation below is a high shine lipstick showcasing the ability of X-TEND™ 226 to impart shine by nature of its high refractive index. This formulation was made by first preparing a color grind using a roller mill on melted Ganex® WP-660 to which color mix is added before milling. The Phase A ingredients were mixed and heated to 90-95° C. until uniform, and cooled to 82-85° C. Then, Phase B was added with mixing until homogeneous. Next, Phase C color grind was added and mixed for 30 minutes. Finally, the Phase D ingredients were added and mixed until uniform. The contents were poured into a lipstick mold at 82-85° C. and solidified. When applied onto lips, a brilliant shine and glossy appearance radiated from the lips of the user.

Brilliant Shine Moisturizing Lipstick

| Ingredients | % w/w |
|---|---|
| Phase A | |
| Ozokerite (White Ozokerite Wax 170) | 10.50 |
| Polyethylene (Performalene Polymer) | 5.00 |
| Octyldodecyl Stearate (CERAPHYL ® ODS) | 13.00 |
| Diisopropyl Adipate (CERAPHYL ® 230) | 2.00 |
| Octyldodecyl Stearoyl Stearate | 12.00 |
| (CERAPHYL ® 847) | |
| Phenethyl Benzoate (X-TEND ™ 226) | 4.00 |
| C₁₂₋₁₅ Alkyl Lactate (CERAPHYL ® 41) | 11.00 |
| Myristyl Lactate (CERAPHYL ® 50) | 1.00 |
| Hydrogenated Polyisobutene | 6.10 |
| (Panalane L-14E) | |
| VP/Eicosene Copolymer (GANEX ® V-220) | 5.00 |
| VP/Hexadecene Copolymer (GANEX ® V-216) | 5.00 |
| Disodium Lauriminodipropionate | 0.20 |
| Tocopheryl Phosphates (VITAL ™ ET) | |
| Retinyl Palmitate (Vitamin A Palmitate) | 0.10 |
| Phase B | |
| Isopropylparaben (and) Isobutylparaben (and) | 0.40 |
| Butylparaben (LIQUAPAR ® OIL) | |
| Phase C | |
| Isocetyl Stearoyl Stearate (CERAPHYL ® 791) | 3.28 |
| Tricontanyl PVP (GANEX ® WP-660) | 0.07 |
| Red 7 Lake (C6507 D&C Red #7 CA Lk) | 2.35 |
| Iron Oxides (C33-5198 Black Iron Oxide) | 0.75 |
| Blue 1 Lake (FD&C Blue #1 Aluminum Lake) | 0.25 |
| Phase D | |
| Mica (Mica UF) | 8.00 |
| Bismuth Oxychloride (Pearl Glo UVR) | 3.00 |
| Mica (and) Iron Oxides (Cloisonne Gold) | 4.00 |
| Mica (and) Iron Oxides | 3.00 |
| (Cloisonne Super Rouge) | |
| | 100.00% |

### EXAMPLE 2 (Control)

The lipstick formulation of Example 1 was prepared, however, without including X-TEND™ 226 therein. When it was applied to the lips of the user, a rather dull appearance resulted.

The use of the phenylethyl esters of the invention in hair care compositions to enhance shine is illustrated by the following examples.

### EXAMPLE 3

The formulation was made by charging water to a suitable mixing vessel with sweep blade agitation. Styleze® W-20 and Liquid Germall® Plus were added with mixing, which was continued until homogeneous. Then, X-TEND™ 226 was added and mixed until uniform. Finally, Rapi-Thix® A-60 was added and the batch was mixed until uniform (about 15 minutes).

The hair care composition of Example 3 was applied to both wet and dry hair. A high shine appearance on the hair resulted in both cases.

High Shine Hair Care Composition

| Ingredient | Wt. % |
|---|---|
| Water | 91.55 |
| Polyquaternium-55 (Styleze ® W-20) (ISP) | 1.25 |
| Propylene Glycol (and) Diazolidinyl Urea (and) | 0.50 |
| Iodopropynyl Butylcarbamate | |
| (Liquid Germall ® Plus (ISP) | |
| 2-Phenylethyl Benzoate (X-TEND ™ 226) (ISP) | 5.00 |
| Sodium Polyacrylate (and) Hydrogenated | 1.70 |
| Polydecene (and) Trideceth-6 (Rapi-Thix ® A-60) | |
| (ISP) | |
| | 100.00% |

### EXAMPLE 4 (Control)

The formulation of Example 3 without X-TEND™ 226 also was applied to hair. A dull appearance resulted.

While the invention has been described with particular reference to certain embodiments thereof, it will be understood that changes and modifications may be made which are within the skill of the art. Accordingly, it is intended to be bound only by the following claims, in which:

What is claimed is:

1. A method of imparting high shine to a polymeric substrate which comprises applying thereto a phenylethyl or benzyl ester, or substituted benzyl ester.

2. A method according to claim 1 wherein said ester is an ester of 2-phenylethyl alcohol, 1-phenylethyl alcohol or benzyl alcohol.

3. A method according to claim 1 wherein said ester is 2-phenylethyl benzoate, 2-phenylethyl toluate, di-2-phenylethyl phthalate, 1-phenylethyl benzoate or benzyl benzoate.

4. A method according to claim 1 wherein the spectral reflections of light on said substrate are substantially enhanced by the presence of said ester thereon.

5. A method according to claim 3 wherein the refractive index of said ester is about 1.5.

6. A method according to claim 1 wherein said substrate is hair or skin.

7. A method according to claim 1 wherein said substrate is leather, imitation leather or a plastic.

8. A composition for imparting high shine, gloss or luster to a polymeric substrate which includes a phenylethyl ester, benzyl ester or substituted benzyl ester.

9. A composition according to claim 8 which is a personal care composition.

10. A personal care composition according to claim 8 which is a lipstick.

11. A personal care composition according to claim 8 which is a hair care product.

12. A personal care composition according to claim 7 wherein said substrate is leather, imitation leather or a plastic.

13. A personal care composition according to claim 8 in which said ester is the reaction product of an aryl carboxylic acid and a phenylethyl alcohol, benzyl alcohol or a substituted benzyl alcohol.

14. A personal care composition according to claim 8 in which said ester is 2-phenylethyl benzoate, 2-phenylethyl toluate, di-2-phenylethyl phthalate, 1-phenylethyl benzoate or benzyl benzoate.
